(19) United States
(12) Patent Application Publication
(10) Pub. No.: US 2009/0318825 A1
(43) Pub. Date: Dec. 24, 2009

(54) INVESTIGATING NEUROLOGICAL FUNCTION

(76) Inventor: Kerry Kilborn, Renfrewshire (GB)

Correspondence Address: MYERS BIGEL SIBLEY & SAJOVEC, PO BOX 37428, RALEIGH, NC, 27627, US

(21) Appl. No.: 12/307,804

(22) PCT Filed: Jul. 9, 2007
(86) PCT No.: PCT/GB2007/002557

§ 371 Date: Aug. 21, 2009

(30) Foreign Application Priority Data
Jul. 7, 2006 (GB) 0613551.1

(51) Int. Cl. A61B 5/0402

(52) U.S. Cl. 600/544

(57) ABSTRACT

This invention relates to a method and apparatus for identifying degenerative disorders and particularly to the early and accurate diagnosis of Alzheimer's Disease.

### FIELD OF INVENTION

This invention relates to a method and apparatus for identifying degenerative disorders and particularly to the early and accurate diagnosis of Alzheimer's Disease.

### BACKGROUND OF INVENTION

Alzheimer's Disease (AD) is a progressive degenerative disorder of cognitive function (memory). Early diagnosis of AD will enable preventative treatment to start at an early stage of the disease. A conclusive diagnosis of AD is not possible without post mortem brain samples. Current methods for diagnosing AD in elderly patients therefore involve a clinical assessment by a specialist and the use of questionnaires or other tools to assess cognitive function. The diagnosis of AD is based on excluding other conditions that could be causing the clinical symptoms (e.g. vascular disease, brain tumour).

The Primary Degenerative Dementias, such as Alzheimer's Disease and Vascular Dementia, are unfortunately common and associated with a significant morbidity and mortality and is at present one of the major challenges to the clinician and the health services. It is estimated that 26% of women and 21% of men over the age of 85 have some form of dementia and that in England and Wales there are 700,000 with some form of this disorder. As our populations age this problem will inevitably increase as the risk of dementia increases exponentially with increasing age.

The majority of these illnesses develop insidiously over a number of years and in the early ‘prodromal’ phases diagnosis can be difficult. Many older people also show deterioration in memory function which may, or may not, progress to dementia and a plethora of terms has been developed to categorise this group, such as Age Associated Memory Impairment (AAMI) and Mild Cognitive Impairment (MCI). These are likely to be rather heterogeneous groups and at the present time their natural history is poorly known.

Faced with these difficulties, the recent development of treatment strategies (e.g. Anticholinesterases) and preventative strategies (e.g. neuroprotective agents, vitamin E, and possibly statins) has brought the present unsatisfactory status of early diagnosis into clearer focus. This difficulty will become more important if current studies (which are nearing completion) show that anticholinesterases can indeed hinder the progression of the Mild Cognitive Impairment of old age into AD. In addition to permitting earlier intervention in patients who have AD, more accurate early diagnosis could help avoid the risks of inappropriate treatment for those patients who do not.

Present tools available to assist clinical diagnosis are often not helpful in early diagnosis. These are either images of brain structure (e.g. CT or MRI scan) or of brain function (e.g. SPECT or EEG). Structural imaging is unlikely to be valuable at this stage in the illness when structural changes are likely to be extremely modest or absent and again merge into the spectrum of changes seen with ageing. Functional imaging holds the most promise but the presently available measures do not reliably pick up deficits at the level required.

It is an object of at least one aspect of the present invention to obviate or mitigate at least one or more of the aforementioned problems.

It is a further objective of at least one aspect of the present invention to provide a method for providing early and accurate detection of neurological disorders such as Alzheimer's Disease.

### SUMMARY OF THE INVENTION

According to a first aspect of the present invention there is provided a method for diagnosing a neurological disorder which comprises:

a cognitive task;

collecting electroencephalogram (EEG) signals from a person conducting said cognitive task;

and conducting an analysis of said EEG signals to form an algorithm which is capable of being used to determine if the person conducting the cognitive task has a neurological disorder.

In particular, the neurological disorder which may be determined in this method may be Alzheimer's Disease. The method may therefore be used to provide an aid to early and accurate detection of Alzheimer's Disease.

Typically, the cognitive task may be a cognitive probe task which may be computerised and may be a simple two-part choice task in which patients are asked to decide whether each test stimulus has been presented before or not. Typically, the stimuli may consist of coloured line drawings in combination with clearly spoken words. Responses are made by pressing either a “yes” or “no” button on a response box. Preferably, the computerised cognitive task assesses the short-term associative memory of a patient.

In the cognitive task, the computer may present stimulus pairs consisting of a picture and a spoken word. For example, the image may depict a train, and the spoken word may be “tunnel”.

For example, a list of suitable images and spoken words are as follows:

| Image | Spoken Word |
|---|---|
| baby | blanket |
| tiger | beast |
| palette | paint |
| brush | artist |
| duck | egg |
| eagle | fly |
| feet | walk |
| hand | clean |
| heart | soul |
| coat | rain |
| glove | ice |
| Hat | boy |
| button | shirt |
| dress | doll |
| boot | toe |
| barrel | sugar |
| basket | story |
| bath | towel |
| bottle | wine |
| bowl | breakfast |
| bucket | coal |
| Jug | honey |
| purse | thief |
| wallet | salary |
| Cat | mouse |
| Dog | bone |
| turtle | tail |
| goat | cheese |
| horse | harness |
| Pig | farm |
| sheep | wolf |
| bread | baker |
| cake | wedding |
| chicken | Roast |
| onion | Cry |
| corn | Butter |
| apple | Pie |
| cherry | Blossom |
| lemon | Slice |
| pear | Market |
| pineapple | Fruit |
| chair | Library |
| cradle | Newborn |
| desk | Writer |
| cook | Stove |
| stool | Bar |
| Bed | Pillow |
| tooth | Dentist |
| Eye | Wink |
| nose | Cold |
| Ear | Ring |
| clock | Time |
| pencil | Write |
| pipe | Smoke |
| telephone | Friend |
| trolley | Shop |
| umbrella | Wind |
| watch | Arm |
| camera | Actress |
| Fan | Fever |
| sink | Kitchen |
| fork | Knife |
| oven | Turkey |
| spoon | Soup |
| glass | Milk |
| Pan | Bacon |
| candle | Wax |
| lamp | Table |
| anchor | Sailing |
| crane | Tower |
| bell | Kitten |
| chain | prisoner |
| Pin | Sharp |
| airplane | Ticket |
| Car | Driver |
| rocket | Planet |
| submarine | Dive |
| tractor | Trailer |
| train | Tunnel |
| drum | Band |
| guitar | String |
| piano | performer |
| trumpet | Tune |
| violin | Concert |
| mountain | Ski |
| feather | Light |
| present | birthday |
| coin | Golden |
| flag | Emperor |
| lightning | Cloud |
| moon | Star |
| medal | military |
| postcard | Smile |
| flower | Bee |
| palm | Island |
| rose | Plant |
| tree | Willow |
| leaf | Bush |
| book | paragraph |
| newspaper | Read |
| Bat | Cave |
| fish | Hook |
| monkey | Wild |
| rabbit | Clover |
| snake | Bite |
| Ant | queen |
| fountain | freeze |
| bridge | river |
| door | Key |
| fireplace | chimney |
| house | window |
| tent | student |
| gate | estate |
| pool | Swim |
| Axe | Fire |
| hammer | Nail |
| ladder | Roof |
| rake | autumn |
| Saw | blade |
| shovel | Dirt |
| drill | engineer |
| puppet | Film |
| ball | beach |
| dice | Game |
| bicycle | accident |
| boat | fisherman |
| wagon | Road |
| canoe | Lake |
| arrow | warrior |
| cannon | castle |
| sword | knight |
| spear | throw |
| nest | Bird |
| Web | spider |
| elephant | heavy |
| Fox | Park |
| lion | jungle |
| bear | Fur |
| seal | circus |
| antelope | Zoo |

The same stimulus pairs may then be presented once or twice again in a short time interval or a long time interval. The short time interval may, for example, be five intervening items (e.g. 20 seconds) and the long interval may be about thirty-nine intervening items (e.g. 156 seconds). The patient being tested decides whether the image and the spoken word have been presented for the first time (i.e. new) or has been presented before (i.e. old). The patient therefore may press either of two buttons for “new” or for “old”.

During the cognitive probe task a multichannel EEG (e.g. a 128 or 256 channel) is performed on a test subject. This may take the form of a dense array EEG which may be applied in the form of a “geodesic sensor net” over a patient's head. The dense array EEG may detect the patient's brain electrical activity (i.e. the EEG signals) for each stimulus of an image and a spoken word. The collected signals of EEG may be detected in the form of event-related potentials (ERPs). An ERP is an electrophysiological response by the brain to a stimulus which reflects brain operations involved in processing a stimulus. In practice, the robustness of the ERP is enhanced by the presentation of numerous stimuli of a particular type, and the resulting time-locked EEG signal is averaged to cancel out noise, allowing the brain's response to the stimulus to stand out clearly. The time point at which the stimulus is presented is recorded together with the time course of the EEG, enabling the isolation of the portion of the EEG signal (the “epoch”) which corresponds in time to the processing of the stimulus. These stimulus-locked time regions, when averaged, are referred to as Event-Related Potentials, or ERPS.

Preferably, the EEG collecting apparatus may be a 128-channel geodesic sensor net.

The memory performance of a patient may therefore be determined by analysing the responses obtained. A specific time interval may be selected using the event-related potentials. For example, the time region of interest, with regard to the cognitive ad brain events, is 2 seconds beginning at the onset of the stimulus presentation.

Typically, it may also be found that specific channels in the EEG collecting apparatus may provide improved results. For example, the mean ERP across 4 channels in the geodesic sensor net as used herein (channels 17, 18, 22, 23) may show a large and reliable difference which may serve as a clinically useful electrophysiological marker which differentiates between healthy controls and patients diagnosed with AD. Also, this difference may be observed maximally during a time interval from 384 to 440 milliseconds. Preferably, particular channels are therefore selected to collate EEG data from.

Typically, a mean event-related potential may be chosen over which data may be measured to determine if a patient has a neurological disorder such as Alzheimer's Disease. This makes the measurement easier.

Typically, the obtained data may be formatted into an algorithm which may be plotted on a graph of behavioural measure obtained from signal detection theory against mean event-related potentials. For classification purposes, the data of the possible AD and the matched control subjects may be modelled using logistic regression.

The dependent variable was group (possible AD, matched control) and the independent variables were memory d′, response latency, difference ERP between long delay and new items, and the mean ERP of all items. Independent variables whose coefficient was not significant according to the Wald test were removed from the model. The variables that remained in the model were the memory d′ (Chi-Square(1)=13.73, p<0.001) and the mean ERP (Chi-Square(1)=5.63, p<0.05).

The algorithm may be in the form of a sloped straight line whereupon on one side of the line substantially all persons or at least 70-90% have Alzheimer's Disease and on the other side substantially all or at least 70-90% do not have Alzheimer's Disease.

Preferably, the algorithm for Alzheimer's Disease has an estimated logit of:

5.78−1.88×memory d′−0.41×mean event-related potential>0.

According to a second aspect of the present invention there is provided use of the method according to the first aspect in diagnosing a neurological disorder in a patient.

Preferably, the neurological disorder may be Alzheimer's Disease.

According to a further aspect of the present invention there is provided apparatus for diagnosing a neurological disorder comprising:

computerised means for displaying a visual stimulus and means for emitting an audible signal;

a response box comprising a ‘NEW’ and ‘OLD’ button; and

an EEG array.

### BRIEF DESCRIPTION OF THE DRAWINGS

Embodiments of the present invention will now be described, by way of example only, with reference to the accompanying drawings in which:

FIG. 1a represents t-values from possible patients with Alzheimer's Disease and matched controls of mean event-related potentials collected at channels 104 and 29 in a 128-channel geodesic sensor net;

FIG. 1b represents t-values from patients with possible Alzheimer's Disease and matched controls of the mean event-related potentials of all items collected at channel 18 of a 128-channel geodesic sensor net.

FIG. 2 represents a 128-channel geodesic sensor net;

FIG. 3a represents the mean difference event-related potentials between channels 104, 105, 110, 111 and 28, 28, 34, 35 in a long delay;

FIG. 3b represents mean event-related potentials of all items, averaged across channels 17, 18, 22 and 23; and

FIG. 4 represents the mean event-related potential amplitude measures for chosen time periods and the behavioural measures for patients with possible Alzheimer's Disease and their age match controls.

### DETAILED DESCRIPTION

### General Description

The present invention relates to a diagnostic tool for the early detection of neurological disorders such as Alzheimer's Disease (AD). The diagnostic tool employs a dense array EEG combined with a cognitive task. Dense array EEG is a measure of brain electrical activity at very high spatial and temporal resolutions. These measures are combined with a cognitive task which taps into mental functions known to be vulnerable in the early stage of AD. The invention is designed to provide positive information about the likely presence of changes in cognitive and brain function consistent with a diagnosis of potential AD.

### Functional Components

The invention comprises three main functional components.
A computerised cognitive task. This is a simple two-part choice task in which patients are asked to decide whether each test stimulus has been presented before or not. Stimuli consist of coloured line drawings paired with clearly spoken words. Responses are made by pressing either a “new” or “old” button on a response box. The task is structured in two 9 minute blocks, with practice trials to start each block.
Dense array EEG. The present invention employs a 128-channel EEG system to acquire brain electrophysiology data while a patient carries out the cognitive task. The EEG system (obtained from Electrical Geodesics Inc.) applies sensors in a “geodesic sensor net”. Electrodes are encased in sponges, and held in place in a gentle tension network by thin elastic threads. Prior to application to a patient, the sponges are soaked in a warmed solution of saline and baby shampoo. The damp sponges provide the necessary contact with the scalp. No abrasion or gels are required. Set up and application takes 5-10 minutes. During this time the patient is seated in a comfortable chair. The sensor net is lightweight, and the only possible mild discomfort is from damp hair.
Automated analysis. Software will carry out an automated analysis and report procedure. The analysis is based on an empirically derived algorithm (explained below). The algorithm produces a classification based on both EEG data and behavioural data.

### Rationale for Device Design

Changes in cognition are important early hallmarks of AD and other dementias. Current tests in wide clinical use measure some aspects of cognitive function. However, such tests are not capable of tapping into cognitive events as they unfold in real time. This means that a range of uncontrolled factors can influence results, such as strategies or individual differences in behaviour, reducing the usefulness of such tests. This problem is particularly difficult at relatively early stages of Alzheimer's Disease, where changes in cognition due to incipient pathology merge into the spectrum of normal ageing.

The present invention addresses this issue by combining a syndrome-specific computerised cognitive task with a time-sensitive measure of brain function.

The cognitive task is designed to assess performance in the domain of episodic memory. Episodic memory involves the recollection of specific events. The formation of new episodic memories requires the hippocampus, a region of the brain in the medial temporal lobe. The pathology of AD is known to specifically affect the hippocampus in the early stages of the disease. As such, episodic memory is vulnerable in the early stages of AD.

The hippocampus is also known to play a central role in the coordination and combination of information from different sensory modalities, in particular from auditory and visual inputs. For this reason, the cognitive task incorporates stimulus pairs consisting of one visual and one auditory stimulus. Thus the cognitive probe task is designed to stimulate hippocampal functions including formation of new memories and integration of visual and auditory information. Both kinds of hippocampal function may be vulnerable to disruption by AD pathology, and together aspects of these functions are the subject of measurement and comparison in the present invention.

Psychological studies have shown that cognitive, and hence brain, events can be measured in milliseconds. This is especially true for cognitive operations which are normally automatic in nature, such as understanding a word, recognising a face, or shifting attention from old to new information. AD causes damage to brain regions that must normally function in concert, disrupting the critical of different brain and cognitive involved in apparently simple tasks. The onset and degree of such disruption can be assessed by time-sensitive cognitive tasks carried out in combination with a sensitive measure of brain function.

In order to measure changes in cognitive operations due to early stage AD, the present invention employs Event-Related Potentials, or ERPs. ERPs are averaged epochs of brain electrical activity (EEG) which are time-locked to the presentation of a stimulus. Research suggests that tests of cognitive function (e.g. memory attention, spatial orienting) using ERPs may detect changes in AD earlier than other techniques. In addition, because it is possible to accurately relate scalp distributions of ERP effects to underlying generators, it may be possible to discriminate AD from conditions that do not have the same underlying pathology but do mimic early overt symptoms.

The present invention uses a 128-channel digital EEG system. This contrasts with the 12-20 sensor systems used for most clinical EEG. The advantages of 128 EEG sensors are easy to explain. Brain electrical events produce potential fields, which spread and contract rapidly across regions of scalp. With inter-sensor distances of 1.5-2 cm, even sharply changing gradients (spikes) can be detected. This provides information about brain function and cognition with an unparalleled degree of temporal and spatial resolution.

### Clinical Information

This section sets out the method and data analysis of a clinical investigation according to the present invention. The focus of the analysis is a logistic regression model which uses EEG combined with behavioural data to assess the diagnostic tool's performance.

### EEG

EEG data collected using a 128-channel Geodesic sensor net. This device allows the rapid and comfortable application of a “dense array” of sensors to the scalp, without the use of gels or abrasion. Set-up and application takes 5-10 minutes.

The data are collected continuously during a test session; a session lasts about 50 minutes. This is broken up into smaller chunks of time corresponding to two blocks. All subjects receive the blocks in the same order. A brief instructions and practice period precedes each block.

The fixed order is necessary to permit eventual comparison of single subject data with group data.

### Associative Memory

In the Associative Memory AM task, the computer presents stimulus pairs consisting of a picture and a spoken word. For example, the image may depict a train, and the spoken word is “tunnel”. At two intervals—short (e.g., 5 intervening items with a total time interval of 20 seconds) and long (e.g. 39 intervening items with a total time interval of 156 seconds), some stimulus pairs are presented for a second and third time. The first (i.e. new) and second (i.e. short interval) presentations are treated in the later analysis as “study” items, while the third presentation is treated as the “test” item. The subject must attend to each pair and decide whether the pair is presented for the first time (new), or has been presented before (old). The decision is registered with a button press on each trial, left index finger for “new” and right for “old”. During performance of the task, dense array EEG is recorded continuously for later analysis, in addition to responses.

There are three independent variables, organised into two blocks:

| | Block 1 | Block 2 |
|---|---|---|
| First Study (new items) | 50 | 50 |
| Second Study (repeated at short interval) | 39 | 39 |
| Test (repeated at long interval) | 30 | 30 |
| Total 238 trials. | | |

Each trial ran according to the following schedule:

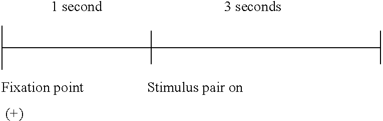

The visual stimulus appears at the same time the auditory stimulus begins. The auditory stimuli are of course variable in length, but have been controlled for length and word frequency. The visual stimulus remains on screen for three seconds. The subject's response must occur during this three second interval while the stimuli are presented. Items receiving a response with the right index finger are counted for that subject as “old”, and items receiving no response are counted as “new”.

### EEG Sampling and Pre-Processing

The EEG is sampled at 250 Hz, or one sample each 4 msec, at each of 128 sensors. The time region of interest, with regard to the cognitive and brain events, is 2 seconds beginning at the onset of the stimulus presentation. This ultimately yields 128×500 samples for each of 3 conditions, after averaging.

These stimulus-locked time regions, when averaged, are referred to as Event-Related Potentials, or ERPs.

Each subject's data are treated as follows to derive values for further analysis:
1. Segmentation, to isolate the time-locked 2 second epoch of EEG data per trial.
2. 20 Hz Filter, to remove 50 Hz line noise.
3. Eyeblink correction.
4. Artefact rejection.
5. Average by condition (New/Short/Long).
6. Bad channel replacement.
7. Re-reference to average over all channels
8. Baseline correction.

### Clinical Investigations

### a) Design

The clinically relevant subject groups are defined according to diagnostic categories drawn from The National Institute of Neurological and Communicative Disorders and Stroke (now called NINDS)-Alzheimer's Disease Related Disorders Association (now called Alzheimer's Association) (NINCDS-ADRDA) Criteria. These criteria have been validated both clinically and pathologically. In brief, the categories are:

1. Possible Alzheimer's Disease (POAD)

2. Probable Alzheimer's Disease (PrAD)

3. Unlikely Alzheimer's Disease

All patient volunteers were consecutive referrals to Royal Alexandra Hospital memory clinic in Paisley, or participants in another project. Clinical diagnosis according to standard criteria was carried out by specialist clinicians.

All subjects were screened to exclude possible co-morbid conditions which may influence initial assessment relevant to AD. A non-clinical control group of healthy older individuals was also recruited. However, because it was considered important to avoid amplifying differences by creating artificially homogeneous groups, the only further criterion applied was age.

The aim of the diagnostic tool according to the present invention is to provide early and accurate detection of AD. For this reason the main focus of the analysis is a comparison between healthy controls of a similar age and patients who meet the clinical criteria POAD. The other patient groups are included in the analysis, but the PoAD group includes some patients who, because of the more advanced state of the disease, and less uncertainty in the clinical diagnosis, may not normally be candidates for an early diagnostic test.

The final assignment of patients to relevant clinical groups was based on a complete diagnostic work-up which took place over several weeks. This led to the testing of patients who were eventually classed as ‘Unlikely AD’ and as ‘PrAD’ as well as ‘PoAD’. Although the cognitive task was specifically designed as a test for PoAD, the inclusion of other clinical subjects who do not belong to the target group offered an opportunity for an unbiased test of the diagnostic tool's performance.

### b) Results

### Demographic

The participants in the study are described below:

Controls:
67 Normal control participants were recruited in local newspapers, members of local bowling clubs, respondents to a brochure sent to members of clubs for the elderly, and controls from another project. The data of two participants could not be used because of excessive eye movements. The remaining were 34 female and 31 male participants. Their mean age was 70.3 years (range 59 to 95 years).

Patients:
Possible AD. 26 participants with POAD were recruited from the Royal Alexandria Hospital in Paisley and from another project. One participant was rejected because of excessive eye movements. The remaining were ten female and 15 male participants. Their mean age was 76.1 years (range 63 to 89 years).
Unlikely AD. Six participants were recruited who were admitted to the Royal Alexandria Hospital with memory problems, but were diagnosed as probably not having AD. They were one female and five male participants.
Probable AD. 13 participants with PrAD were recruited from another project. One participant could not do the Associative Memory task. Two participants did not complete the test and stopped after the first block of the Transfer Task. The data of two participants could not be used because of excessive eye movements. The remaining were four female and four male participants. Their mean age was 75.3 years (range 63 to 84 years).
Other conditions. Five people from the other project were tested, but their data was not used because they had various other diagnoses, such as Alcoholism, Leaning Disabilities, Multiple Sclerosis, and Lewy Body Dementia. Four people from the other project were tested who were admitted, but they were not diagnosed as having AD. Their data was not used because their Clinical Dementia Rating was 0 (no dementia). (The six ‘no AD’ participants from the Royal Alexandria Hospital all had a CDR of 0.5 (questionable dementia)).

The primary comparison involves the healthy controls and patients diagnosed as having possible AD. The two groups were initially not matched for age (t(88)=3.55, p<0.001). It is known that both memory and ERP measures are affected by age which could therefore be a confounding variable. To eliminate this potential confound, the control group was split into 31 participants of 70 years or older (the ‘matched controls’) and 34 participants of 69 years or younger (the ‘young controls’). The mean age of matched controls was 76.0 and the mean age of the young controls was 65.6 years. The matched controls did not differ significantly from the participants with possible AD (t(63)−0.07, n.s.).

### Memory Performance

Memory performance for the behavioural data was analysed from the ‘old’ responses to items presented at a long delay and new items, using the d′ measure from signal detection theory. The statistic d′ is a useful way of describing the performance of individuals on a performance task (such as the cognitive probe task described above) in which individuals make decisions under conditions of uncertainty. In our case, individuals are required to respond “new” or “old” according to whether they decide a particular stimulus has been presented before or not. Individuals who suffer from a deficit in episodic memory such as may be caused by early stages of AD are likely to experience more uncertainty in making decisions under these conditions. The d′ statistic is used to measure and describe this aspect of performance on the cognitive probe task.

The mean latency across all types of items was also computed. This mean latency may be related to retrieval efficiency, because the better people can search their memory, the faster they may respond, be it positively or negatively. However, participants were not asked to respond as fast as they could because that may have caused anxiety and, therefore, the response latency might be quite noisy. Another problem with relating response latency with retrieval efficiency is that the latency would also be affected by memory strength.

The d′ of the controls was 4.07 (standard deviation (sd) of 0.62) and the d′ of the participants with possible AD was 1.99 (s.d. 1.36), which were statistically different (t(54)=7.61, p<0.0001). The mean response latency of the controls was 1057 msec. (s.d. 159) and of the possible AD's 1223 msec. (s.d. 365), which were also statistically different (t(54)=2.29, p<0.05).

The Event Related Potential (ERP) data were analysed for two conditions. The first condition was the difference between the ERP's recorded for items presented at the long delay and the short delay. These difference ERP's were expected to reflect the effects of memory processes. The second condition was the mean ERP across all types of items. Participants were asked to indicate for each item whether it was presented before. Therefore, it was expected that the second condition would reflect retrieval processes.

### Selection of Spatial Locations by ERP Effect

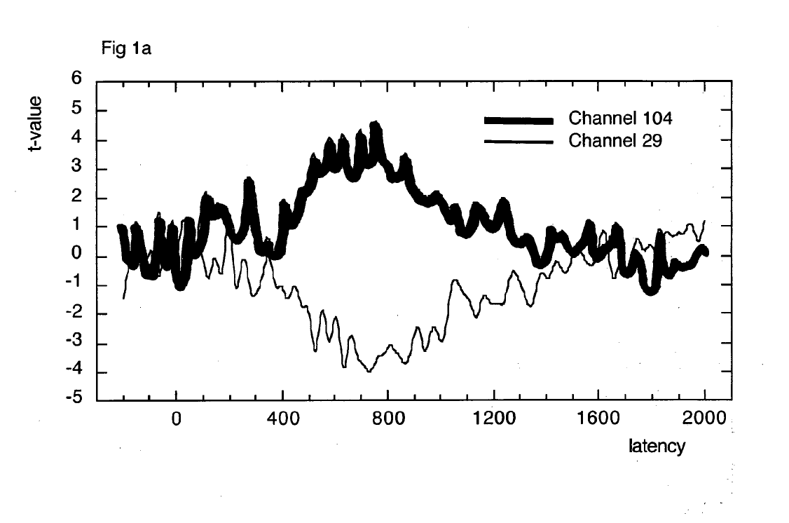

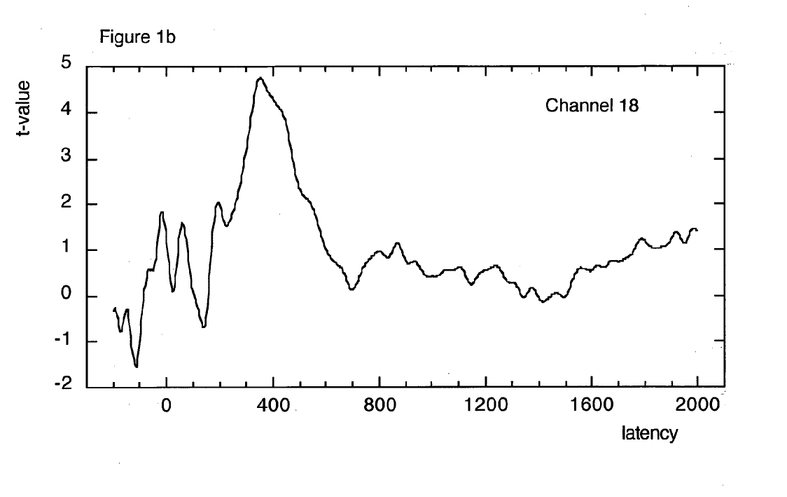

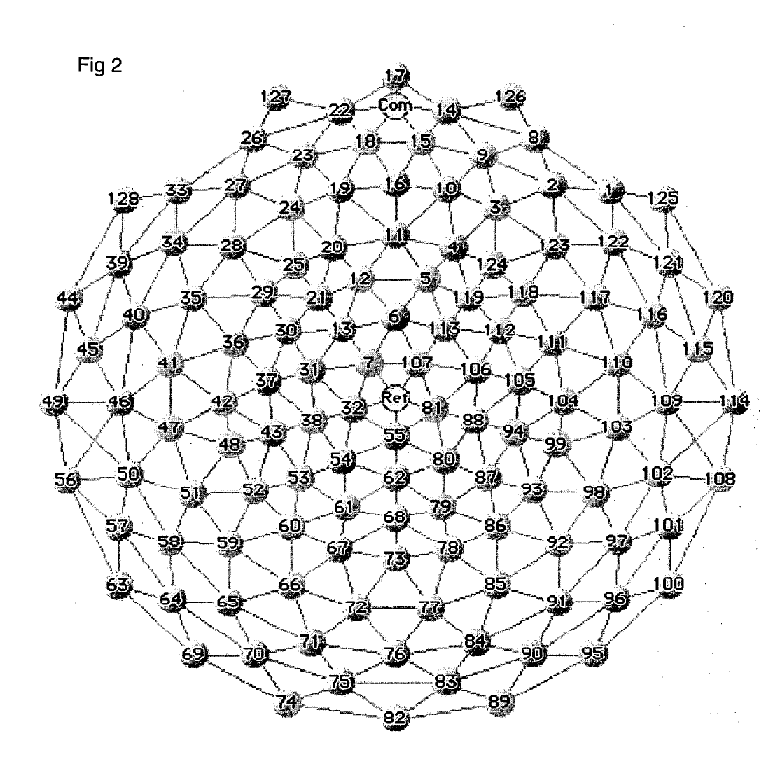

The channels for the ERP analyses were selected using graphs of the t-value of the difference between the participants with possible AD and the controls. The effect for the difference between long delay and new items showed a dipole and therefore, the difference between positive and negative channels will be used. Examples of channels with a positive and a negative effect are shown in FIG. 1a. A group of 4 positive channels were used (channels 04, 105, 110 and 111, see FIG. 2) and 4 negative channels (28, 29, 34 and 35) to allow for individual anatomical differences and slight variations in the fit of the electrode net on the participants head. The effect on the mean ERP across all conditions did not show a clear dipole and, therefore, only four positive channels (17, 18, 22, 23) were used. An example of these channels is given in FIG. 1b. (Note: spatial location selection was not based on results form PrAD, unlikely AD, or young controls).

FIG. 2 shows the geodesic sensor now comprising a 128 channel map used to obtain the experimental results.

### Selection of Time Interval by ERP Effect

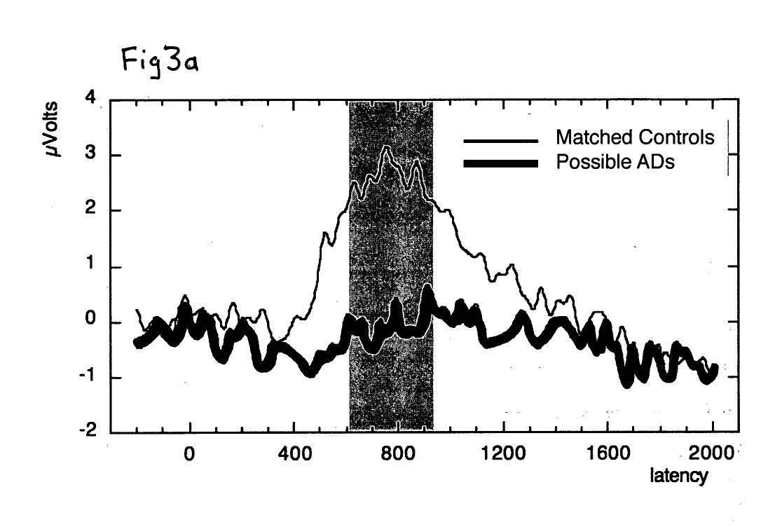

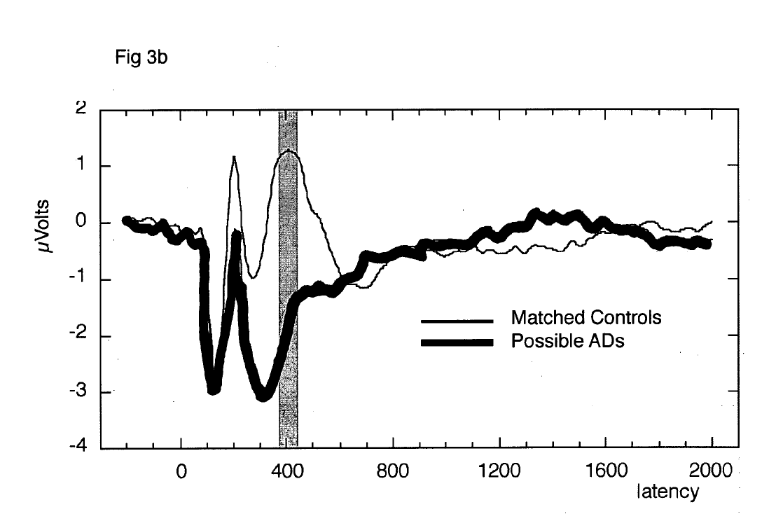

A selection of the mean ERP across a certain time interval was used for further analyses. The mean difference ERPs between the long delay and new items for possible AD and control participants are shown in FIG. 3a. The mean ERP in the shade time interval (between 608 and 944 msec) was used, which is the interval for which the control ERP was larger than 2.1 μVolt. (Note: time intervals were not based on results from PrAD, unlikely AD, or young controls).

FIG. 3b shows mean ERPs of all items averaged across channels 17, 18, 22 and 23.

The validity of the selected ERP measure was assessed by correlating them with their respective behavioural measures across all five groups. Analysis of covariance was used to do this, with participant group (young control, matched controls, unlikely AD, possible AD, PrAD) as a factor and memory d′ (for the difference ERP) or response latency (for the mean ERP) as covariates.

### Correlation of ERP and Behavioural Data

The interaction between the memory d′ and participant group was not significant and was removed from the analyses of the difference ERP. The only significant effect on the difference ERP was that of the memory d′ (F(1,98)=16.88, p<0.0001). The effect of participant group was no longer significant (F<1). There was significant effect of subject group on the difference ERP (F(4,94)=3.41, p<0.05), a significant effect of response latency (F(1,94)=5.43, p<0.05) and a significant interaction between group and latency (F(4,94)=2.97, p<0.05). The relationship between response latency and the mean ERP was negative in all groups, apart from the young control participants.

### Classification by Logistic Regression

For classification purposes, the data of the possible AD's and the matched control subjects were modelled using logistic regression (using the StatView statistical package). The dependent variable was group (possible AD, matched control) and the independent variables were memory d′, response latency, difference ERP between long delay and new items, and the mean ERP of all items. Independent variables whose coefficient was not significant according to the Wald test were removed from the model. (The Wald test is a statistical test, typically used to test whether an effect exists or not between two nominal or ordinal variables). The variables that remained in the model were the memory d′ (Chi-Square(1)=5.63, p<0.05). All groups were classified as having dementia according to whether the estimated logit was as follows:

5.78−1.88*memory d′−0.41*mean ERP>0

The resulting classification counts and percentages of correct classification are given in Table 1 for the five participating groups.

TABLE 1
Classification Results

| | Dementia | No Dementia | % Control |
|---|---|---|---|
| Young controls | 0 | 34 | 100 |
| Matched controls | 2 | 29 | 93.6 |
| Unlikely AD | 1 | 5 | 83.3 |
| Possible AD | 23 | 2 | 92 |
| Probable AD | 8 | 0 | 100 |

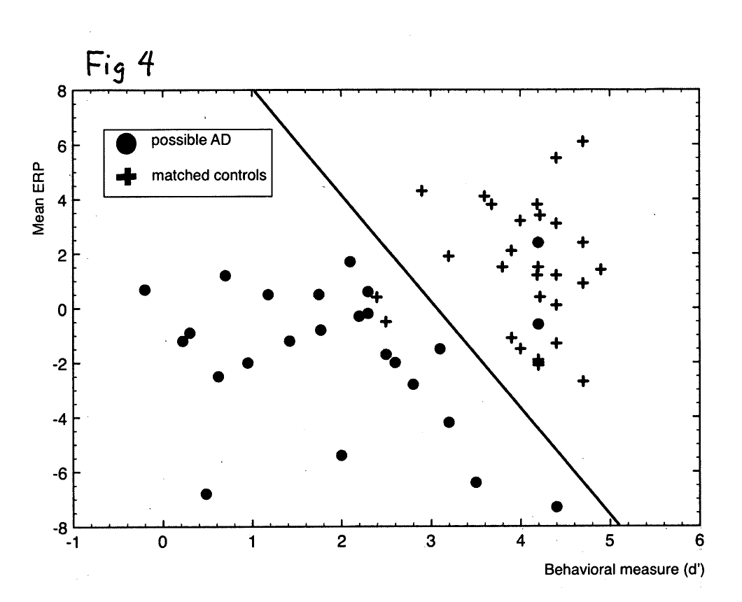

The sensitivity (possible AD) was 92% and the specificity (matched controls) was 93.6%. The relationship between the mean ERP amplitudes in the relevant time periods and the behavioural measures for possible AD and matched controls is shown in FIG. 4.

FIG. 4 is a plot of results of behavioural measure (d′) versus mean ERP. The mean ERP amplitude measures for the chosen time periods and the behavioural measures (d′) for the patients with possible AD and their age matched controls are therefore shown in FIG. 4. Above the sloped line is substantially all of the matched controls, and below the sloped line is substantially all patients with possible AD. If a patient is therefore tested and found to be below the sloped line, it is highly likely that the patient has AD.

The three groups that were not involved in the selection of the ERP channels and time intervals and the logistic regression modelling do not belong to the target group because they were too young (the young controls), their diagnoses was too uncertain (the unlikely AD's) or they were too severe (the PrAD's). However, the model can still be tested using these groups, because they still should give sensible results. Specifically, firstly, fewer young controls than matched controls should be classified as having dementia because it is less likely that there are people with early dementia among the young controls than among the matched controls. Secondly, more PrAD's than PoAD's should be classified as having dementia, because it is less likely that they are misdiagnosed than that the PoAD's are misdiagnosed. Thirdly, the unlikely AD's should be more often classified as not having dementia, under the minimal assumption that trained clinicians are correct more often than not. The model passed all these three tests.

1. A method for diagnosing a neurological disorder which comprises:
a cognitive task;
collecting electroencephalogram (EEG) signals from a person conducting said cognitive task;
and conducting an analysis of said EEG signals to form an algorithm which is capable of being used to determine if the person conducting the cognitive task has a neurological disorder.

2. The method according to claim 1, wherein signals relevant to diagnosis of the neurological disorder are taken from the hippocampus region.

3. A method according to claim 1, wherein the neurological disorder is Alzheimer's Disease.

4. A method according to claim 1, wherein the method provides early and accurate detection of Alzheimer's Disease.

5. A method according to claim 1, wherein the cognitive task is a cognitive probe task which is computerised and is a two-part choice task in which patients are asked to decide whether each test stimulus has been presented before or not.

6. A method according to claim 5, wherein the stimuli consist of coloured line drawings in combination with clearly spoken words.

7. A method according to claim 1, wherein the cognitive task provides stimulus pairs consisting of a picture and a spoken word.

8. A method according to claim 7, wherein pre-determined stimulus pairs are presented at least once again in a short time interval or a long time interval and a patient decides whether the image and spoken word has been presented for the first time or has been presented before.

9. A method according to claim 1 wherein the EEG data is obtained from a multichannel EEG apparatus.

10. A method according to claim 1, wherein a dense array EEG apparatus is applied in the form of a geodesic sensor net over a patient's head.

11. A method according to claim 10, wherein the dense array EEG detects a patient's brain electrical activity for each stimulus of an image and a spoken word.

12. A method according to claim 9, wherein the collected signals of EEG are detected in the form of event-related potentials (ERPs).

13. A method according to claim 9, wherein an EEG collecting apparatus is a 64-channel, 128-channel or 256 channel system design.

14. A method according to claim 13, wherein specific channels of the EEG collecting apparatus are utilised to provide improved results such as channels 17, 18, 22 and 23 of a 128 channel sensor as shown in FIG. 2, or similar regions from other sensor arrays which show a large and reliable difference which serves as a clinically useful electrophysiological marker which differentiates between healthy controls and patients diagnosed with Alzheimer's Disease.

15. A method according to claim 1, wherein particular channels in an EEG collecting apparatus are selected to collate EEG data.

16. A method according to claim 1, wherein a mean event-related potential is chosen over which data is measured to determine if a patient has a neurological disorder such as Alzheimer's Disease.

17. A method according to claim 1, wherein obtained data is formatted into an algorithm which is plotted on a graph of behavioural measure obtained from signal detection theory against mean event-related potentials.

18. A method according to claim 17, wherein the algorithm is in the form of a sloped straight line whereupon on one side of the line substantially all persons have, for example, Alzheimer's Disease and on the other side substantially all of the persons have no neurological disease such as Alzheimer's Disease.

19. A method according to claim 17, wherein the algorithm for Alzheimer's Disease has an estimated logit of:
5.78−1.88×Memory d′−0.41×Mean Event-Related Potential>0.

20.-21. (canceled)

22. Apparatus for diagnosing a neurological disorder comprising: computerised means for displaying a visual stimulus and means for emitting an audible signal:
a response box comprising two input buttons, such as a ‘new’ and ‘old’ button; and
an apparatus capable of obtaining an EEG from a subject.

23. The apparatus according to claim 22, wherein the apparatus capable of obtaining an EEG from a subject is in the form of a multichannel sensor array designed to be worn over the head comprising sensors for detecting brain signals all over the head and in particular from the hippocampus region.
